(19) United States
(12) Patent Application Publication
(10) Pub. No.: US 2005/0197627 A1
(43) Pub. Date: Sep. 8, 2005

(54) Method and system for deploying protective sleeve in intraluminal catherization and dilation

(75) Inventors: Alexander Huang, Menlo Park, CA (US); David Needleman, Sausalito, CA (US); Henry Lao, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PERCUTANEOUS SYSTEMS, INC., Mountain View, CA (US)

(21) Appl. No.: 10/794,337

(22) Filed: Mar. 5, 2004

(51) Int. Cl. A61M 5/00

(52) U.S. Cl. 604/171

(57) ABSTRACT

Protected access through a body lumen or a tissue tract is provided by a protective sleeve. The protected sleeve is initially in a furled configuration and is unfurled by an advancement member extended through the lumen or tract. A catheter or other access member may then be advanced over the advancement member and through the protective sleeve.

### BACKGROUND OF THE INVENTION

1. Field of the Invention

The present invention relates generally to medical apparatus and methods. More particularly, the present invention relates to methods and systems for intraluminal and percutaneous access to body cavities and other target locations.

Catheters, dilators, endoscopes, and other access devices are useful in a very wide variety of diagnostic and therapeutic medical procedures. Such access devices may be introduced through existing body lumens, such as a urethra, a ureter, a blood vessel, a hepatic duct, a cystic duct, a cervical canal, a fallopian tube, and the like, in order to provide an access path from a region external to the patient to a target region within the body. For some target regions, such as blood vessels, at least a portion of the access path will pass transcutaneously and/or through internal tissues in order to reach the body lumen or target region within the body. Such access through solid tissue typically relies on forming a “tissue tract” by passing a stylet, needle, or other penetrating element through the tissue. Once such access is provided, a wide variety of diagnostic techniques may be performed, such as specimen collection, biopsy, and the like. Alternatively, therapeutic procedures, such as endoscopic procedures, drainage procedures, interventional procedures, and the like, may also be performed.

Passage of such access devices through body lumens and/or through tissue tracts in solid tissue presents certain risks and discomfort to the patient. For example, introduction of catheters through the urethra can carry pathogens into the bladder, causing infection. Repeated access through any natural body lumen or tissue tract can also cause trauma to the lumen, placing the patient at risk of injury or infection. Injury to the lumen may also cause bleeding as well as short or long term stricture.

In order to at least partly overcome some of these deficiencies, the use of sheaths or “protective sleeves” has been proposed. For example, in a series of patents assigned to RTC, Inc., of St. Paul, Minn., the use of an everting sleeve initially stored within a catheter to protect the urethra in access procedures is described. The sleeve is attached at its distal end to a ring which can be placed against the entrance to the urethra before the catheter is advanced. As the catheter is advanced, the protective sleeve is pulled from an internal lumen of the catheter and everts around the advancing tip of the catheter so that it is deployed over the urethral wall. Although very promising, the need to evert the sleeve around the blunt tip of the catheter can provide challenges for deployment.

The use of radially expanding sleeves for introducing dilators and other surgical instruments has been proposed in a series of patents originally assigned to Innerdyne Medical, Inc. These patents describe use of a braided tubular sleeve for introducing dilators and other laparoscopic tools. The braid is introduced over a needle, such as an insufflation needle when used in laparoscopic procedures. After introducing the sleeve, the needle is removed, and a dilator introduced through the sleeve to stretch the sleeve and increase the diameter of the sleeve lumen. Use of the sleeve for vascular and other procedures is taught in some of the Innerdyne patents. Although quite useful, particularly for dilation, the Innerdyne braided sleeve has substantial thickness and adds considerably to the deployed diameter of an device through which it is introduced. Such added thickness, high tissue friction, can be a problem, particularly in procedures such as urethral access, where a low profile is desired.

For these reasons, it would be desirable to provide additional and improved methods and systems for introducing diagnostic and therapeutic devices through body lumens and tissue tracts. In particular, it would be desirable to provide improved protective sleeves and methods for deploying protective sleeves during the introduction of such diagnostic and therapeutic devices. For example, it would be desirable to provide sleeves which could be deployed over a device during introduction of that device through a body lumen, where the sleeve does not have to evert about a distal end of the device during deployment. It would be further desirable to provide such sleeves having a very thin wall thickness so that any increase in overall profile of the apparatus being introduced is minimized. It would be further desirable to provide protective sleeves for covering medical access devices, where the sleeves can be maintained in a sterile environment at all times up until deployment of the sleeve into the body lumen. At least some of these objectives will be met by the inventions described hereinbelow.

2. Description of the Background Art

The use of an everting sleeve composed of thin, tensilized polytetrafluoroethylene for introducing catheters to body lumens is described in U.S. Pat. Nos. 5,531,717; 5,676,688; 5,711,841; 5,897,535; 6,007,488; 6,240,968; and EP605427B1. The use of braided and other radially expanding sheaths for introducing surgical tools, and other articles is described in U.S. Pat. Nos. 5,431,676; 5,454,790; 5,814,058; 5,836,913; 6,080,174; 6,325,812; and 6,494,893. The use of sleeves for protecting biopsy needles and cannulas is described in U.S. Pat. No. 4,262,677; DE10031661A1; and Woitzik and Kraus (2003) Surg. Endosc. 17:311-314.

### BRIEF SUMMARY OF THE INVENTION

The present invention provides methods and systems for accessing target locations within a patient's body. The target locations will often be inside of a body cavity or organ but can also be within solid tissue. Exemplary target locations include sites to be diagnosed, such as by biopsy, aspiration, imaging, chemically analysis and the like. Alternatively, the target locations may be intended for therapeutic treatment, such as drainage, drug delivery, ablation, excision, thermal treatment, photodynamic therapy, and the like. Access to the target location will usually be at least partly through a natural body lumen, such as a urethra, a ureter, a blood vessel, a hepatic duct, a cystic duct, a cervical canal, a fallopian tube, or the like. Additionally or alternatively, the access can be wholly or partly through solid tissue. To provide access through solid tissue, it will be necessary to form a tissue tract either before or at the same time as introducing the protective sleeve of the present invention, as will be described in detail below. An exemplary technique employing the protective sleeves of the present invention will be described in connection with transurethral access of the bladder, but it will be appreciated that the principles and embodiments of the present invention may be applied to a much wider variety of target locations and access routes.

In a first aspect, the present invention provides a method for deploying a sleeve through a body lumen. A furled sleeve is first positioned at a luminal entry point where a leading edge of the sleeve is coupled to an advancement member. The advancement member can be any one of a variety of conventional or novel introducing tools, including guidewires, catheters, probes, wires, cannulae, and the like, and the sleeve will typically but not necessarily have an inside diameter which is greater than the outside diameter of the advancement member, typically being at least 25% larger and often at least 50% or more larger. The advancement member may be provided separately from the furled sleeve, in which case the method may further comprise attaching the advancement member to the sleeve prior to unfurling. Alternatively, the advancement member may be pre-attached to the sleeve and sterilely packaged in that condition. In either case, the advancement member is advanced into a desired body lumen, either a natural lumen or a tissue tract, whereby the sleeve is unfurled and deployed into the lumen. Exemplary natural body lumens include a urethra, a ureter, a blood vessel, a hepatic duct, a cystic duct, a cervical canal, a fallopian tube, and the like. In contrast, a tissue tract may be formed using an advancement member having a self-penetrating tip, such as a sharpened tip, an electrosurgical tip, an ultrasonic tip, or the like. The advancement member may thus be passed through solid tissue to either the desired target location or to another body lumen, duct, or cavity, which in turn leads to the target location. The target location may be itself present in solid tissue or within a body cavity, lumen, or duct. In some instances, it will be desirable to pre-form a tissue tract, e.g. using a stylet, an electrosurgical probe, or the like, prior to advancing the advancement member therethrough to deploy the protective sleeve of the present invention.

In a second aspect, the present invention provides methods for accessing a body lumen of a patient. The method comprises providing a furled sleeve and advancing one end of the sleeve through the body lumen causing the sleeve to progressively unfurl. A separate access member is then introduced through an axial passage of the unfurled sleeve while the sleeve remains in the body lumen. Typically, the access member will be a catheter, a dilator, a sheath/stylet combination, an endoscope, or other conventional diagnostic or therapeutic tool. The access member will often be introduced over an advancement member which is used to advance the sleeve through the body lumen, such as a guidewire, small diameter catheter, wire, probe, or the like. The method will be used to access target locations through natural body lumens and tissue tracts, generally as described above with respect to the first method of the present invention. The protective sleeve will have at least one axial passage therethrough, and may optionally have two axial passages, three axial passages, or more. Usually, the advancement member will be positioned in one of the axial passages, where the access member may then be introduced through the same axial passage or through an alternative axial passage if others exist. Alternatively, the advancement member could be attached at the distal end of the protective sleeve but be located over the exterior of the sleeve. In all instances, the advancement member could include a loop, ring, or other expansive element at its distal end which is capable of opening the distal ends of the axial passage(s).

In a third aspect of the present invention, a luminal access system comprises a furled sleeve having at least one axial passage therethrough and an advancement member adapted to couple to a leading edge of the sleeve and to unfurl the sleeve as the advancement member is advanced through a body lumen. The furled sleeve will be positionable at a luminal entry point, such as a natural body orifice or intended site for percutaneous access, and the advancement member may be advanced through the furled sleeve in order to deploy the sleeve into a body and lumen or a tissue tract being created. A preferred furled sleeve will be present in a storage chamber which maintains the sleeve in a sterile and protected condition prior to use. The sleeve may be in a rolled configuration, a folded configuration (typically accordion-like folds), or otherwise reduced in size to minimize the size of the furled sleeve enclosure. The storage chamber will have a slot for passing of the sleeve from the interior to the exterior. Usually, the storage chamber will also have an axial passage to permit advancement of the advancement member through the chamber in order to deploy the sleeve.

Preferred protective sleeves are polymeric tubes, particularly thin-walled polymeric tubes made from a lubricious polymer or a polymer which may be lubricated on at least one side. The polymeric tube typically has a length in the range from 5 cm to 50 cm, preferably from 10 cm to 25 cm, an inner diameter (for a single lumen) in the range from 2 mm to 12 mm, preferably in the range from 2 mm to 6 mm, and a wall thickness in the range from 0.01 mm to 0.05 mm, preferably from 0.08 mm to 0.13 mm. Preferred polymers for the polymeric tube include polytetrafluoroethylene (PTFE), polyethylene (PE), perfluoroalkoxy (PFA), polyurethane (PU), perfluoromethylvinylether (MFA), perfluoropropylvinylether (PPVE), and the like. A preferred polymer comprises a tensilized PTFE/PPVE copolymer.

The advancement member may be pre-attached to the distal end of the protective sleeve or may be adapted to permit attachment immediately prior to the deployment. In either case, it will often be desirable to provide attachment that is frangible so that the sleeve can be detached from the advancement member, e.g. by advancement of an access member over the advancement member, such as advancement of a dilator over a guidewire advancement device. The advancement member will often have an axial lumen through at least a portion thereof to receive a catheter or guidewire.

### BRIEF DESCRIPTION OF THE DRAWINGS

FIGS. 1A-1D illustrate a method and system for deploying a protective sleeve and advancing a dilator through the deployed sleeve in accordance with the principles of the present invention.

FIGS. 2A and 2B illustrate a protective sleeve having a single axial passage therethrough in accordance with the principles of the present invention.

FIGS. 3A and 3B illustrate a protective sleeve having a pair of parallel axial passages in accordance with the principles of the present invention.

FIG. 4 illustrates an advancement member attached to a distal end of the protective sleeve, where the advancement member does not pass through the sleeve.

FIG. 5 illustrates a system and kit constructed in accordance with the principles of the present invention.

FIG. 6A-6D illustrate a method of the present invention for deploying a catheter through a urethra and into the bladder of a patient.

### DETAILED DESCRIPTION OF THE INVENTION

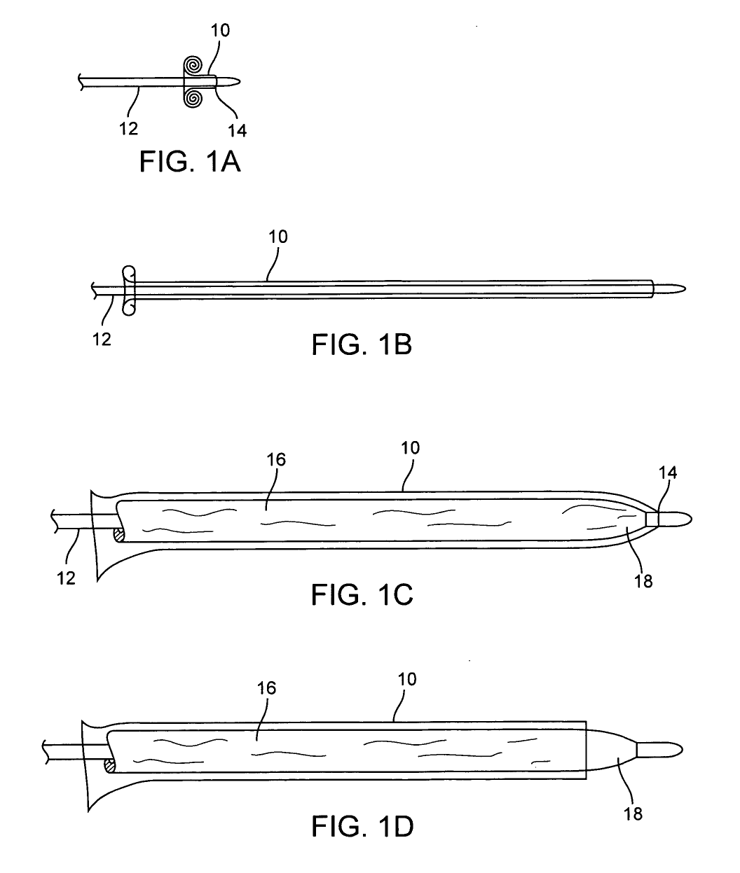

Referring now to FIGS. 1A-1D, the protective sleeve 10 is attached to an advancement member 12 at a location 14 near (but usually not covering) a distal end of the advancement member. The protective sleeve is typically a thin-walled polymeric tube of the type described above. The protective sleeve is furled so that, initially, it does not extend over the length of the advancement member and typically will not extend over a distal end of the tip of the advancement member. As illustrated, the protective sleeve is rolled to form a toroidal geometry. The end of the sleeve which is at the center of the toroid is brought through the center and attached at location 14 to the advancement member 12. Usually the protective sleeve will be pre-attached to the advancement member so that it is ready to use when removed from a sterile package. Alternatively, the protective sleeve 10 and advancement member 12 could be configured to allow the user to make the attachment immediately prior to use, optionally as the advancement member 12 is passed through the interior of the furled sleeve 10. As a further alternative, the advancement member could be configured to automatically engage and capture the protective sleeve as the member is advanced through a storage chamber holding the furled sleeve.

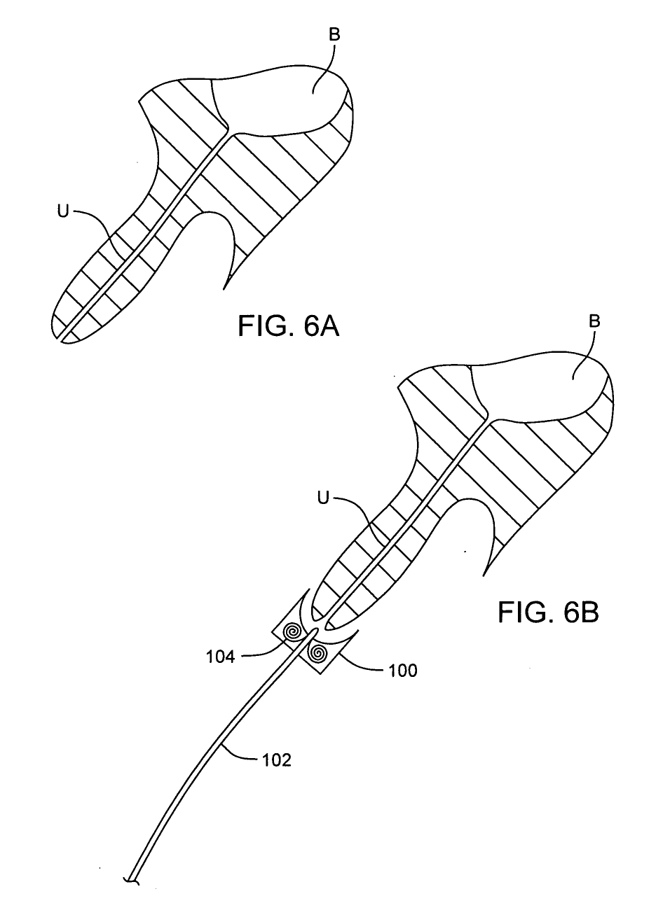

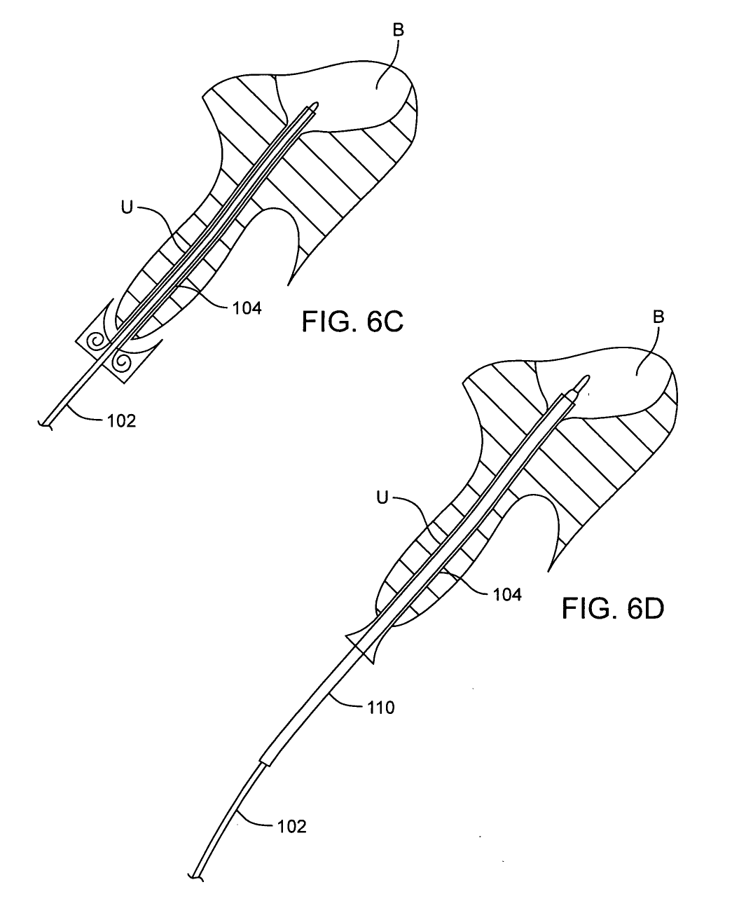

In any case, advancement of the advancement member 12 in an axial direction (to the right in FIGS. 1A-1D), unfurls the protective sleeve 10 as illustrated in FIG. 1B. Upon unfurling, the protective sleeve 10 will typically retain a low profile configuration. This will be advantageous, since the advancement member and sleeve will often be passing through a relatively narrow body lumen or tissue tract, such as through a urethra as described hereinbelow in connection with FIGS. 6A-6D. The distal tip of the advancement member is unconstrained by the sheath so as to facilitate location of, entry to, and navigation through the body lumen.

After the advancement member 14 has been passed through the body lumen or tissue tract to a desired target location, a separate access member, such as a catheter, dilator, sheath/dilator, endoscope, or the like, may be passed through the sleeve 10 (often also widening the body lumen simultaneously) and optionally over the advancement member 12 which may be, for example, a guidewire. As illustrated in FIG. 1C, a dilator 16 may be passed over the advancement member 12. The dilator 16 has tapered distal end 18 which acts to open the width or diameter of the protective sleeve 10 as the dilator is advanced in a direction from left to right in the drawings. During the initial stages of the advancement, the protective sleeve 10 will typically remain attached to the advancement member 12 at attachment point 14. Usually, however, it will be desirable to frangibly attach the sleeve 10 to the advancement member 12 so that passage of the dilator 16 will detach the sleeve from the advancement member, as shown in FIG. 1D. The advancement member 12 may then be withdrawn from the dilator 16 or other catheter or access member. Thus, the lumen through the access member will then be available for a diagnostic, therapeutic, or other procedure.

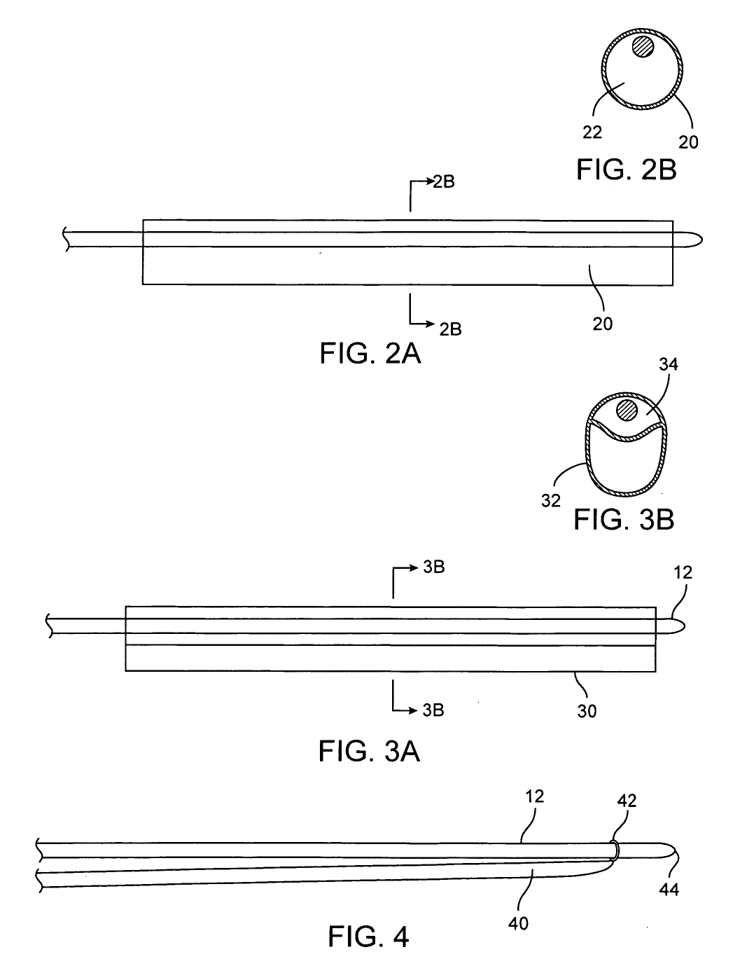

As illustrated in FIGS. 1A-1D, the protective sleeve 10 is a simple tube having a single axial passage. The protective sleeve, however, can have a variety of other configurations. As shown in FIGS. 2A and 2B, a sleeve 20 is again a simple tube having a single axial passage 22. The advancement member 12, however, is shown to be secured to one side of the axial passage 22, thus leaving a major portion of the passage available for the advancement of various access members or other articles.

In FIGS. 3A and 3B, protective sleeve 30 comprises a first central passage 32 and second central passage 34. The advancement member 12 is positioned in the second axial passage, leaving the first axial passage available for introduction of access members or other articles, tools, or the like. It will be appreciated that three, four, five, or more axial passages, could be provided in the protective sleeves in accordance with the principles of the present invention.

In FIG. 4, a protective sleeve 40 is shown attached to an exterior of the advancement member 12. A ring 42 is attached to a distal end of the sleeve 40 and over the outside of the advancement member 12, usually spaced proximally of distal tip 44 of the member 12. Thus, it is not necessary for the advancement member to pass through a central passage or any portion of the protective sleeve in order to deploy it.

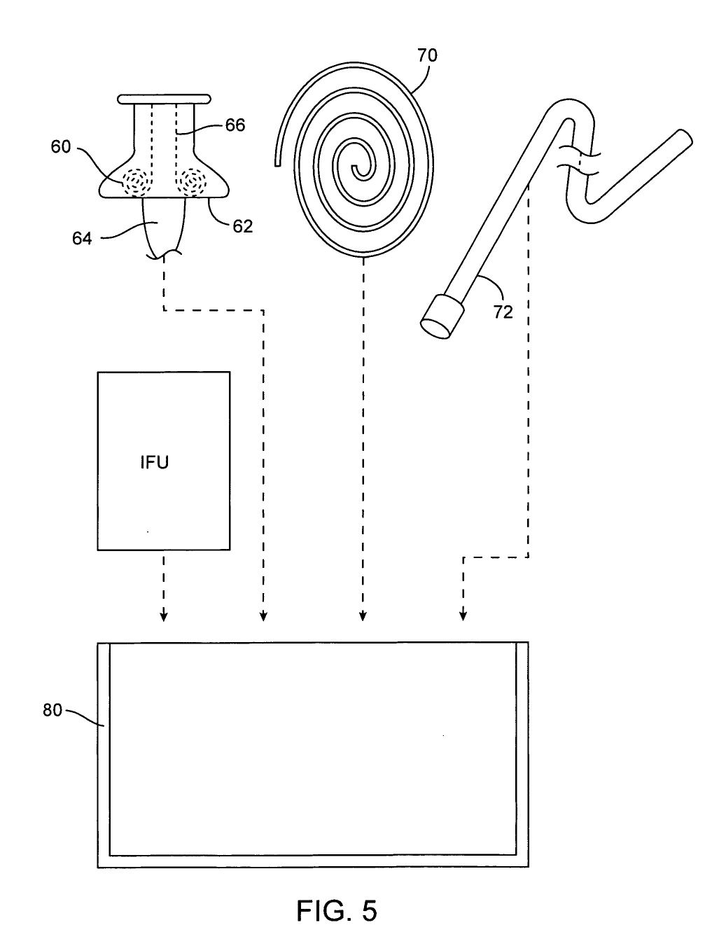

Referring now to FIG. 5, a kit and system according to the present invention comprise a protective sleeve deployment member 60 having a flange surface 62 at one end. A protective sleeve 64 is furled within the flanged portion 62, as shown in broken line, and a central passage 66, also shown in broken line, allows an advancement member to pass through the deployment member 60 in order to engage and advance the sleeve 64. The system further includes an advancement member 70, shown as a guidewire, and an access catheter 72 which is suitable for introduction over the guidewire and through the interior of the protective sleeve 64. Instructions for use setting forth a method in accordance with the present invention will also be provided, and all system components will typically be provided sterilely within a package 80, typically a pouch, box, tube, or other conventional medical device enclosure.

Use of the methods and devices of the present invention for accessing a bladder B through a urethra U is shown in FIGS. 6A-D. Prior to introduction, the urethra U is generally collapsed and non-dilated as shown in FIG. 6A. As an initial step of the present invention, a furled sleeve enclosure 100 is engaged against the entry to the urethra U, as shown in FIG. 6B. An advancement member 102 is attached to the furled protective sleeve 104, and the advancement member and sleeve are advanced through the urethra and into the bladder B as shown in FIG. 6C. The enclosure may then be removed from the furled sleeve, and an access catheter 110 is advanced over the advancement member 102 and through the protective sleeve 104, as shown in FIG. 6D. The advancement member 102 may then be removed from the lumen of the catheter 110 in order to provide transurethral access to the bladder B.

While the above is a complete description of the preferred embodiments of the invention, various alternatives, modifications, and equivalents may be used. Therefore, the above description should not be taken as limiting the scope of the invention which is defined by the appended claims.

1. A method for deployment of a sleeve through a body lumen, said method comprising:
positioning a furled sleeve at a luminal entry point, wherein a leading end of the sleeve is coupled to an advancement member; and
advancing the advancement member into a lumen, whereby the sleeve is unfurled and deployed into the lumen.

2. A method as in claim 1, wherein the advancement member is advanced through a natural body lumen.

3. A method as in claim 2, wherein the natural body lumen is selected from the group consisting of a urethra, a ureter, a blood vessel, a hepatic duct, a cystic duct, a cervical canal, and a fallopian tube.

4. A method as in claim 2, further comprising forming a tissue tract to provide access to the body lumen from a body surface, wherein the advancement member is advanced through the tissue tract and then into the natural body lumen.

5. A method as in claim 1, wherein advancing the advancement member creates the body lumen through solid tissue.

6. A method as in claim 5, wherein the advancement member has a self-penetrating tip.

7. A method as in claim 5, wherein the advancement member is advanced to a target site in solid tissue.

8. A method as in claim 5, wherein the advancement member is advanced to a target site in a body cavity.

9. A method as in claim 1, further comprising creating the body lumen through solid tissue prior to advancing the advancement member.

10. A method for accessing a body lumen of a patient, said method comprising:
providing a furled sleeve;
advancing one end of the sleeve through the body lumen, causing the sleeve to progressively unfurl, and
introducing an access member at least partly through an axial passage of the unfurled sleeve while the sleeve is in the body lumen.

11. A method as in claim 10, wherein the access member is selected from the group consisting of a catheter, a dilator, a sheath dilator, and an endoscope.

12. A method as in claim 10, wherein the sleeve is advanced through a natural body lumen.

13. A method as in claim 12, wherein the natural body lumen is selected from the group consisting of a urethra, a urethra, a blood vessel, a hepatic duct, a cystic duct, a cervical canal, and a fallopian tube.

14. A method as in claim 12, further comprising percutaneously accessing the natural body lumen to form a tissue tract to provide access to the body lumen from a body surface, wherein one end of the sleeve is advanced through the tissue tract and then into the natural body lumen.

15. A method as in claim 10, wherein advancing the advancement member creates the body lumen through solid tissue.

16. A method as in claim 15, wherein the advancement member has a self-penetrating tip.

17. A method as in claim 15, wherein the advancement member is advanced to a target site in solid tissue.

18. A method as in claim 15, wherein the advancement member is advanced to a target site in a body cavity.

19. A method as in claim 10, further comprising creating the body lumen through solid tissue prior to advancing the advancement member.

20. A luminal access system access system comprising:
a furled sleeve having at least one axial passage therethrough, said furled sleeve being positionable at a luminal entry point; and
an advancement member adapted to couple to a leading end of the sleeve and to unfurl the sleeve as the advancement member is advanced through a body lumen.

21. A luminal access system as in claim 20, further comprising a storage chamber having an interior which contains the furled sleeve and a slot for passing the sleeve from the interior as the sleeve is unfurled.

22. A luminal access system as in claim 21, wherein the storage chamber also has a central axial passage for passing the advancement member as said member is unfurling the sleeve.

23. A luminal access system as in claim 20, wherein the sleeve comprises a polymeric tube.

24. A luminal access system as in claim 23, wherein the elongate tube has a length in the range from 5 cm to 50 cm, an inner diameter in the range from 2 mm to 12 mm, and a wall thickness in the range from 0.01 mm to 0.05 mm.

25. A luminal access system as in claim 23, wherein the polymer is a lubricious polymer.

26. A luminal access system as in claim 23, wherein the polymer is lubricated.

27. A luminal access system as in claim 23, wherein the polymer is selected from the group consisting of polytetrafluoroethylene (PTFE), Polyethylene (PE), perfluoroalkoxy (PFA), polyurethane (PU), perfluoromethylvinylether (MFA), and perfluoropropylvinylether (PPVE).

28. A luminal access system as in claim 27, wherein the polymer comprises tensilized PTFE/PPVE copolymer.

29. A luminal access system as in claim 20, wherein the sleeve has at least two axial passages therethrough.

30. A luminal access system as in claim 20, wherein the advancement member is attached to the leading end of the sleeve.

31. A luminal access system as in claim 30, wherein the advancement member is frangibly attached to the leading end of the sleeve so that advancement of an access member through the axial passage of the sleeve breaks the frangible attachment.

32. A luminal access system as in claim 20, wherein the advancement member comprises a guide wire adapted to receive a catheter thereover.

33. A luminal access system as in claim 20, wherein the advancement member comprises a guide catheter having a lumen adapted to receive a catheter or guidewire therethrough.

34. A luminal access system as in claim 20, wherein the advancement member comprises a self-penetrating distal tip and has sufficient column strength to advance said tip through tissue by pushing on a proximal portion thereof.

35. A luminal access system as in claim 20, wherein the advancement member comprises a dilator.

36. A luminal access system as in claim 35, wherein the advancement member comprises a sheath dilator.

37. A luminal access system as in claim 20, further comprising an access member adapted to be advanced through the axial passage of the sleeve after said sleeve has been unfurled in the body lumen.

38. A luminal access system as in claim 37, wherein the access member is selected from the group consisting of a catheter, a dilator, a sheath dilator, and an endoscope.
